(19) United States
(12) Patent Application Publication
(10) Pub. No.: US 2022/0298226 A1
(43) Pub. Date: Sep. 22, 2022

(54) METHACRYLATED COLLAGEN

(71) Applicant: JELLAGEN LIMITED, Cardiff (GB)

(72) Inventors: Andrew Mearns SPRAGG, Cardiff (GB); David WILLIAMS, Cardiff (GB)

(21) Appl. No.: 17/619,939

(22) PCT Filed: Jun. 18, 2020
(86) PCT No.: PCT/GB2020/051468

§ 371 Date: Dec. 16, 2021

(30) Foreign Application Priority Data
Jun. 18, 2019 (GB) 1908709.7

(51) Int. Cl. C07K 14/78; A61L 27/24; A61L 27/52; A61L 27/26; A61L 27/54; A61K 9/19

(52) U.S. Cl. CPC C07K 14/78; A61L 27/24; A61L 27/52; A61L 27/26; A61L 27/54; A61K 9/19

(57) ABSTRACT

The present invention provides a novel collagen methacrylamide, methods by which the collagen methacrylamide may be manufactured and uses for said novel collagen methacrylamide, such as 3D-printing, in the treatment and healing of wounds, as a cosmetic and in regenerative medicine.

### FIELD OF THE INVENTION

The present invention relates to a novel collagen methacrylamide, methods by which the collagen methacrylamide may be manufactured and uses for said novel collagen methacrylamide.

### BACKGROUND OF THE INVENTION

Collagen is the most ubiquitous protein in the mammalian proteome, comprising up to 30% of all proteins (Leitinger & Hohenester, 2007). It forms a large part of the extracellular matrix and connective tissue, offering strength and flexibility to tissues in the body. Besides its role in mechanical strength, collagen functions as a signalling molecule, regulating cellular migration (Greenberg, et al., 1981), differentiation (Bosnakovski, et al., 2005) and proliferation (Pozzi, et al., 1998), and functions in haemostasis (Farndale, et al., 2004).

One form of collagen which is commonly used is the collagen hydrogel. Collagen hydrogels consist of a network of soluble collagen fibres that are prevented from dissociating by polymer entanglement and/or covalent cross-linking. They can also be formed from colloidal suspensions. The physical properties of hydrogels can be tuned depending on the route of manufacture. This topic is covered by several detailed reviews (Drury & Mooney, 2003) (Hennink & van Nostrum, 2012) (Hoffman, 2012).

Collagen hydrogels are rapidly becoming an essential component of modern cell culture techniques. They enable the growth of cells in a 30 lattice more reminiscent of their in vivo environment. The ability to culture cells in vitro in an in vivo environment has obvious benefits for the study of cell biology. 3D hydrogels can also be used as tissue engineering scaffolds, and as such there is a particular interest in the use of collagen as a bio-ink for 3D printing or solid free-form fabrication to provide materials for end applications such as in regenerative medicine, wound healing, or for in cosmetics.

Various attempts at utilising collagen in 3D-printing have resulted in a lack of success. For example, one system which used mammalian type I collagen showed good cell viability but failed to maintain mechanical integrity, a crucial characteristic required for applications such as tissue scaffolds. In response to this obstacle, many groups have trialled the suitability of hybrid materials, only to find that both the biomaterial and the synthetic material was present throughout the scaffold, resulting in undesirable properties, such as low cell attachment. Subsequently, efforts focussed on modifying the collagen itself; however, reaction conditions in these methods often result in unwanted gelation or partial denaturation, resulting in the loss of the collagen's ability to self-assemble into fibrils similar to native collagen.

The patent U.S. Pat. No. 8,658,711 discloses a method of manufacturing collagen methacrylamide from type-I bovine collagen which retains its ability to self-assemble. However, despite bovine collagen being widely used, there are a myriad of disadvantages associated with using this type of collagen. Firstly, it is well known that the use of mammalian collagen in tissue engineering is associated with considerable risk of disease and virus transmission. Secondly, the purification of collagen from mammalian sources is associated with considerable expense, and contaminant molecules carried over from purification methods impair the reproducibility of mammalian collagen hydrogel formation. The latter can significantly compromise the reliability of mammalian collagen products when used in cell culture, and further adds yet another variable to consider in the analysis of experimental data.

Thus, a collagen methacrylamide, derived from an alternative source that lacks the disadvantages described above, suitable for use in a variety of applications, such as 3D-printing, wound healing and regenerative medicine, would be highly desirable.

### SUMMARY OF THE INVENTION

This invention is based on the entirely unexpected finding that, despite the vastly different physicochemical and amino acid properties of jellyfish collagens to mammalian collagen (FIG. 1), jellyfish collagen can be methacrylated to create a product with desirable properties which are particularly suitable for its application in 3D-printing, in the treatment and healing of wounds, as a cosmetic and in regenerative medicine.

Accordingly, a first aspect of the present invention provides for a collagen methacrylamide, characterised in that the collagen is derived from a non-mammalian source. In a preferred embodiment, the collagen is derived from a jellyfish.

A second aspect of the present invention provides for various uses of the collagen methacrylamide, including for use in 3D-printing of tissue or cellular scaffold and tissue models, treatment and healing of wounds, as a cosmetic or in regenerative medicine.

A third aspect of the present invention provides for a method for manufacturing the collagen methacrylamide, wherein the method comprises the following steps: i) reacting methacrylic acid with a carboxylic acid activating reagent in the presence of a carbodiimide to form a methacrylic acid with an activated carboxylic acid group; and ii) reacting free amino groups on the collagen with the activated carboxylic acid groups on said methacrylic acid to form a collagen methacrylamide, or wherein the method comprises reacting free amino groups on the collagen with aminoethyl methacrylate in the presence of a carboxylic acid activating reagent and a carbodiimide.

A fourth aspect of the present invention provides for a collagen methacrylamide formed by the aforementioned method.

A fifth aspect of the present invention provides for a bio-ink comprising the collagen methacrylamide of the present invention.

### BRIEF DESCRIPTION OF THE FIGURES

The invention is illustrated by the following figures, in which:

FIG. 1 shows the comparison between the amino acid composition of bovine collagen and the amino acid composition of jellyfish collagen.

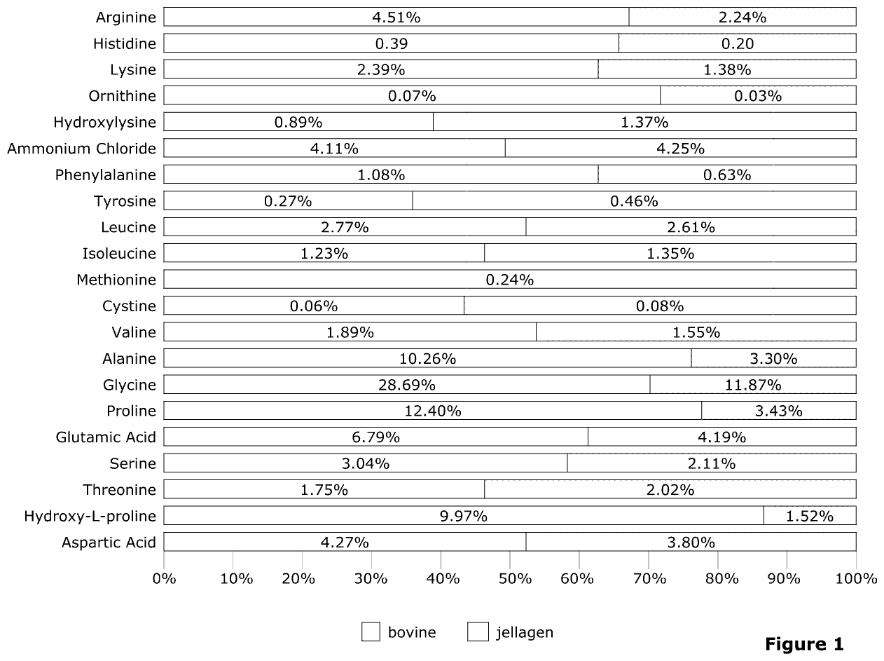

FIG. 2 shows the comparison using Fourier Transform Infrared (FTIR) spectral analysis of methacrylated collagen made according to Condition A (25 mg/mL Aminoethyl methacrylate; 5 mg/mL EDC; 0.5 mg/mL NHS), Condition B (10 mg/mL Aminoethyl methacrylate; 2.5 mg/mL EDC; 0.25 mg/mL NHS), or Condition C (5 mg/mL Aminoethyl methacrylate; 5 mg/mL EDC; 0.5 mg/mL NHS) and non methacrylated collagen.

FIG. 3 shows the comparison using Fourier Transform Infrared (FTIR) spectral analysis of methacrylated collagen made according to Condition A (25 mg/mL Aminoethyl methacrylate; 5 mg/mL EDC; 0.5 mg/mL NHS) and non methacrylated collagen.

### DETAILED DESCRIPTION OF THE INVENTION

The following detailed description contains numerous specific details of the present invention in order to provide a thorough understanding of said invention. It will be understood by those of ordinary skill in the art that embodiments of the present invention may be practiced without these specific details whilst still remaining within the scope of the claims.

The collagen methacrylamide described herein is able to self-assemble from a liquid macromer solution into a fibrillary hydrogel at physiological pH and temperature in a manner similar to that of using a mammalian collagen, but without the associated disadvantages, whilst minimising the denaturation of the collagen protein. This finding is particularly unexpected given the distinctly different chemical nature of jellyfish collagen compared to, for example, bovine collagen. Accordingly, the skilled person in the art would not expect the properties of a mammalian collagen to be replicated in a collagen derived from a non-mammalian source. Yet further, the skilled person would not expect that even if such an alternative source was available, that the use of such a collagen would overcome many of the shortcomings associated with the use of a mammalian collagen, resulting in a variety of superior end products.

A first aspect of the invention provides for a collagen methacrylamide, wherein the collagen is derived from a non-mammalian source. The term ‘non-mammalian’ is intended to specifically exclude collagens derived from mammalian sources, for example, bovine or porcine collagen. Preferably, the non-mammalian source from which the collagen is derived is a jellyfish and will undergo subsequent ‘isolation’ or ‘purification’ to separate the desired collagen from the surrounding anatomical milieu.

There are multiple methods for ‘isolating’, or ‘purifying’ the jellyfish collagen from the anatomical milieu. Many of these will be well known and routine to the skilled person. For example, collagen can be purified from jellyfish by acid extraction, whereby different anatomical parts of the jellyfish are bathed in an acidic solution. ‘Bathing’, or ‘bathed’, refers to the process of incubating the jellyfish in the acid solution for a sufficient amount of time in order to liberate the collagen molecule. An alternative method of collagen purification is enzyme extraction, whereby the jellyfish is incubated with at least one proteolytic enzyme for a sufficient amount of time and under conditions that favour the degradation of the anatomical milieu in order to liberate the collagen molecule. The exact temperature, pH and incubation time of the enzyme extraction method will vary depending on the proteolytic enzyme used. The most suitable conditions will be well known to the skilled artisan. By way of non-limiting example, the enzyme pepsin can be incubated with jellyfish under acidic conditions in order to liberate the collagen molecule. It is envisaged that any enzyme can be used in the enzyme extraction method, and the above examples are intended to be in no way limiting.

The collagen may further be isolated, or purified, from the undesired contaminants of the acid or enzyme extraction method by a number of different means. For example, insoluble contaminants can be removed by centrifugation. If a more pure source of collagen is required, the isolated collagen can be subjected to gel filtration, or an alternative chromatographic method that would enable the purification of the collagen molecule for other soluble contaminants of the extraction process. The exact method of further purification is not particularly limiting. Any method well known and routinely used by a protein biochemist could be adapted for the purpose of obtaining purified, or isolated, jellyfish collagen. This step can also enable the transfer of the jellyfish collagen into the desired storage buffer in order to obtain the desired solution of purified jellyfish collagen. This can be achieved by first equilibrating the chromatographic apparatus with the desired storage buffer before purification. There exist many alternative, well known methods that could be used for this purpose.

In a preferred embodiment, the jellyfish from which the collagen is derived is selected from the group comprising: The order Rhizostomeae and including but not limiting to Rhizostomas pulmo, Rhopilema esculentum, Rhopilema nomadica, Stomolophus meleagris, Cassiopea sp. (upside-down jellyfish), the order Semaeostomeae with examples including Aurelia sp., and other species such as Nemopilema nomurai, or any combination thereof. Preferably, the collagen is derived from Rhizostomas pulmo. Accordingly, the collagen may comprise at least 20%, at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97% at least 98%, at least 99% Rhizostomas pulmo collagen.

It is envisaged that following the isolation and purification processes that collagen fibril formation occurs. The term ‘fibril formation’, or ‘fibrillogenesis’, refers to the process by which collagen molecules undergo controlled aggregation to formation higher order, well-structured macromolecular assemblies. Collagen in vivo is a predominantly extracellular protein whose aggregation into fibrillar structures provides architectural support for surrounding tissues and/or components of the extracellular matrix. The aggregation of collagens, in particular mammalian collagens, is a well-known phenomenon. Different isoforms of mammalian collagens preferentially aggregate into different macromolecular structures. The unique macromolecular structures formed from each collagen isoform is governed by the physicochemical properties of the collagen polypeptide and the conditions under which fibrillogenesis is promoted.

In a preferred embodiment, the collagen methacrylamide may be cross-linked. In the context of the present invention, the term ‘cross-linked’ refers to the linkage of two independent collagen molecules via a covalent bond. Preferably, the collagen molecules to be cross-linked are in the form of collagen fibres, resulting in inter-fibril cross-linking occurring.

In a preferred embodiment, the collagen methacrylamide is provided in a lyophilised or hydrogel form. The concentration of the collagen methacrylamide may be between 0.001% and 100%. It is understood that the desired concentration of the collagen methacrylamide may depend on the end application of the product.

In another preferred embodiment, the form the collagen methacrylamide takes, e.g. lyophilised or a hydrogel, may further comprise another polymer, activating agent, growth factor, antimicrobial compound and/or a therapeutic pharmaceutical compound. The skilled person will recognise how these additional components could be useful when working with cellular or tissue scaffolds and models. Exemplary growth factors for this purpose may include epidermal growth factor (EGF), keratinocyte growth factor (KGF), granulocyte-colony stimulating factor (GCSF), hepatic growth factor (HGF), interleukin-6 (IL-6), interleukin-8 (IL-8), platelet derived growth factor (PDGF), fibroblast derived growth factor-2 (FGF-2), leukemia inhibitory factor (LIF), transforming growth factor β1 (TGF-(β1), transforming growth factor β3 (TGF-β3), vascular endothelial growth factor (VEGF), nerve growth factor (NGF) and/or insulin-like growth factor 1 (IGF-1). Additionally, various antimicrobial compounds may be utilised in order to minimise the loss of valuable cells, reagents, time and effort due to contamination by bacteria, yeast, fungi or mycoplasma. Examples of such compounds include amphotericin B, ampicillin, erythromycin, gentamycin, kanamycin, neomycin, nystatin, penicillin-streptomycin, polymyxin B, tetracyclin, thiabendazole and/or tylosin. Yet further, the resulting collagen methacrylamide form may contain a therapeutic pharmaceutical compound, either as a component of a wound dressing for example or as a drug screening tool. Examples of therapeutic compounds may include vitamins, minerals, natural oils, phytochemicals, enzymes, anti-oxidants, anti-ageing agents, alpha hydroxyacids, glycolic acid, salicylic acid, anti-tumour agents, anti-inflammatory agents, non-steroidal anti-inflammatory agents (NSAIDS), neurotropic agents and the like. It is understood that any of the aforementioned compounds may be used in combination with one another and the particular set of compounds required may be dependent on the desired outcome, for example, the cell type to be cultured.

In a preferred embodiment, the collagen methacrylamide is stable at a temperature of from 15° C. to 80° C., more preferably wherein the collagen methacrylamide is stable at up to at least 37° C. ‘Stable’ means that the collagen methacrylamide does not substantially denature under the given conditions and maintains its desirable properties.

In a second aspect it is envisaged that the collagen methacrylamide of the present invention may be suitable for use in a variety of applications; for example, for use in 3D-printing of tissue or cellular scaffolds and tissue models. It is envisaged that the use of collagen methacrylamide in 3D-printing would be particularly useful for cell culture protocols, whether it be for basic research purposes or as a drug screening tool. Examples of possible cell types which may be cultured include, but are not limited to, chondrocytes, keratinocytes, fibroblasts, adipocytes, osteocytes, keratocytes, lamellar cells, osteoblasts, osteoclasts, macrophages, monocytes, nerve cells, skin cells, stem cells, endothelial cells, kidney cells and hepatocytes. Additionally, it is envisaged that the collagen methacrylamide may be used in the treatment and healing of wounds to create a scaffold for new cells to grow, for example, for use on pressure sores, transplant sites, surgical wounds, ulcers and burns. Accordingly, it is envisaged that the collagen methacrylamide may be present in combination with additional substances, for example, NSAIDs such as Ibuprofen. Furthermore, the collagen methacrylamide may be use as a cosmetic, for example, dermal fillers, or for use in regenerative medicine. The term ‘regenerative medicine’ refers to a specific branch of translation medicine in tissue engineering and molecular biology which aims to develop methods to regrow, repair or replace damaged or diseased cells, organs or tissues. Accordingly, the collagen methacrylamide described herein may be configured as skin, bone tissue, blood vessels, fascia, connective tissue, cartilaginous tissue, ligaments or tendons for example.

In a third aspect, the present invention provides a method for manufacturing the collagen methacrylamide, wherein the method comprises the following steps: i) reacting methacrylic acid with a carboxylic acid activating reagent in the presence of a carbodiimide to form a methacrylic acid with an activated carboxylic acid group; and ii) reacting free amino groups on the collagen with the activated carboxylic acid groups on said methacrylic acid to form a collagen methacrylamide. Alternatively, the method for manufacturing the collagen methacrylamide may comprise reacting free amino groups on the collagen with aminoethyl methacrylate in the presence of a carboxylic acid activating reagent and a carbodiimide.

The method for manufacturing the collagen methacrylamide may further comprise the steps of i) removing excess reagents from said collagen methacrylamide, ii) reacting free carboxylic acid groups on said collagen methacrylamide with a carboxylic acid activating reagent in the presence of a carbodiimide to form a collagen methacrylamide with activated carboxylic acid groups; and iii) reacting said activated carboxylic acid groups on said collagen methacrylamide with aminoethylmethacrylate in the presence of a carbodiimide to form a collagen methacrylamide amidoethylmethacrylate.

The method for manufacturing the collagen methacrylamide herein disclosed results in a photocrosslinkable jellyfish collagen methacrylate, the latter of which is highly biocompatible, easily reproducible, has low immunogenicity and an excellent safety profile, all whilst maintaining all the desirable properties of a mammalian collagen.

In a preferred embodiment, the carboxylic acid activating reagent is selected from the group comprising of: N-hydroxysuccinimide (NHS), N-hydroxysulfosuccinimide (Sulfo-NHS), Hydroxybenzotriazole (HOBt), 1-Hydroxy-7-azabenzotriazole (HOAt), pentafluorophenol and methyl N-(triethylammoniumsulfonyl)carbarnate. However, any carboxylic acid activating reagent which would achieve the effect of forming a methacrylic acid with activated carboxylic acid groups would be suitable.

In another preferred embodiment, the carbodiimide is selected from the group comprising of: 1-ethyl-3-(3-dimethylamino-propyl) carbodiimide (EDC), N,N′-dicyclohexylcarbodiimide (DHC), N,N′-diisopropylcarbodiimide (DIC), 1-(3-dimethylaminopropyl)-3-ethyl carbodiimide hydrochloride, N-cyclohexyl-N′-(2′-morpholinoethyl)carbodiimide-metho-p-toluene sulfonate, N-benzyl-N′-(3′dimethylaminopropyl-carbodiimide hydrochloride, 1-ethyl-3-(3-dimethyl-aminopropyl) carbodiimide methiodide, N-ethylcarbodiimide hydrochloride. However, any carbodiimide which would achieve the desired effect would be suitable.

Visco-elastic properties can be further modified with introduction of cross-linking agents such as genipin, riboflavin, glutaraldehyde (GD), grape seed extract (GSE) and epigallocatechin-3-gallate.

Examples of additional methacrylation agents which may be utilised in the synthesis of methacrylate collagen include glycidyl methacrylate, methacrylic anhydride and methacryloyl chloride.

Jellyfish collagen can be modified to create the collagen methacrylamide herein disclosed by combining one of the aforementioned carboxylic acid activating reagent and one of the aforementioned carbodiimide in MES buffer, in order to activate the carboxyl group of methacrylic acid, for between 1 and 10 minutes at between 15 and 37° C. This solution can subsequently be added to the jellyfish collagen in acetic acid and reacted for 24 hours at 4° C. before being dialysed against acetic acid, lyophilised for 72 hours and re-suspended in acetic acid, Confirmation of the creation of collagen methacrylamide can be verified using proton NMR and quantification of the free amines present before and after the reaction analysed using a trinitrobenzenesulfonic acid (TNBSA) assay.

For subsequent hydrogelation of the collagen methacrylamide, a solution of methacrylated jellyfish collagen at any concentration may be used. The methacrylated jellyfish collagen may be neutralised using a neutralisation buffer. By way of a non-limiting example only, the neutralisation buffer may comprise 10× phosphate buffered saline (PBS) and sodium hydroxide (NaOH). The composition of PBS will be well known to a person skilled in the art. The neutralisation buffer may further comprise UV or visible light photoinitiators. Examples of UV photoinitiators may include benzoinethers, benzilketals, α-dialkoxy-acetophenones, α-hydroxy-alkylphenones, α-amino-alkylphenones, acyl-phosphine oxides, benzophenones, benzoamines, thioxanthones, thioamines, ruthenium(bpy)3, 2, 4, 6-trimethylbenzoyl phosphine oxide or diphenyl (2, 4, 6-trimethylbenzoyl) phosphine oxide. Examples of visible light photoinitiators may include titanocenes, flavins, lvocerin, Irgacure 2959 and naphthalimide derivatives. The jellyfish collagen methacrylamide composition aforementioned may be printed at room temperature and subsequently gelled at 37° C. using irradiation from an appropriate light source.

In a preferred embodiment, the aforementioned method further comprises the step of cross-linking the collagen methacrylamide with a ‘cross-linking agent’ or ‘cross-linker’, Although a variety of cross-linking agents may be used for this purpose, preferably, the cross-linking agent is poly(polyethylene glycol) or light of any wavelength. Preferably, the wavelength of light is in the ultraviolet, blue or visible spectrum. The poly(polyethlylene glycol) may be homobifunctional and be of any molecular weight with, for example, N-hydroxysuccinimide or isothiocyantae being an end functionality of said poly(polyethlylene glycol).

The term ‘cross-linking agent’ or ‘cross-linker’ refers to an agent that can, under certain conditions, form covalent linkages between two independent molecules. In the context of the present invention, a cross-linking agent is used to covalently link two independent collagen molecules. Preferably, the collagen molecules to be cross-linked are in the form of collagen fibres. Preferably inter-fibril cross-linking takes place. In some instances, the cross-linking agents are typically composed of two or more reactive functional groups linked together by a hydrocarbon chain. The two or more functional groups do not necessarily have to be the same. The length of the hydrocarbon chain can also be varied to control the distance between the functional groups. The exact length of the hydrocarbon chain in the context of the present invention is not intended to be limiting.

The ability of the collagen methacrylate to be cross-linked, for example by UV light, allows for the present invention herein described to display flexibility in terms of the level of stiffness of the collagen methacrylate. The degree of stiffness of the end product can be controlled by the amount of exposure to UV light (or alternative cross-linking agent). This control mechanism may be particularly useful in circumstances where stiffness is only desired once the product is positioned correctly, or where the stiffness is determined according to the optimum survival conditions of a specific cell type.

In another preferred embodiment, the collagen methacrylamide produced via the aforementioned method has at least 0.01% of the collagen free amino acid or acid groups acrylated. For example, the collagen methacrylamide may have at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% or 100% of the collagen free amino acid groups acrylated. It is envisaged that any remaining free amino acid or acid groups may be functionalised in a different manner in order to modulate the degree of stiffness and/or visco-elastic properties of the resulting collagen methacrylamide. Alternatively, the free amino acid or acid groups may remain free.

In yet a further preferred embodiment, the pH of the aforementioned method is pH 7.4. The pH of said method may be modulated by the addition of alkaline or acidic substances such as sodium hydroxide or hydrochloric acid respectively. The resulting collagen methacrylamide can subsequently be purified, for example by dialysis, diafiltration, or liquid chromatography (LC) techniques, such as fast protein liquid chromatography (FPLC) or high performance liquid chromatography (HPLC).

In a fourth aspect, the present invention provides for the collagen methacrylamide formed by the aforementioned method. The collagen methacrylamide may or may not have some or all of the features of said collagen methacrylamide herein described.

In a fifth aspect, the present invention provides for a bio-ink comprising the collagen methacrylamide herein described.

The term ‘bio-ink’ refers to a substance comprising living cells that can be used for 3D printing of cellular and tissue scaffold and complex tissue models. Materials that can be used as a bio-ink are intended to mimic an extracellular matrix environment to support the adhesion, proliferation and differentiation of living cells. Bio-inks are processed under much milder conditions compared to that of the more traditional manufacturing materials (e.g. thermoplastic plastics, ceramics and metals) due to the necessity to preserve compatibility with living cells and prevent degradation of bioactive molecules. Accordingly, it is a surprising discovery that jellyfish collagen, given its different physicochemical properties to mammalian collagen, would be successful in this particular application.

In summary, the inventors have created a novel collagen methacrylate which overcomes the disadvantages of mammalian collagen and maintains desirable properties, the manufacture of which can be used in various applications, including 3D-printing, the treatment and healing of wounds, as a cosmetic and in regenerative medicine.

The present invention is now further described with reference to the below example and studies.

### EXAMPLE 1

### Methacrylation of Jellyfish Collagen

Materials

Material required for the methacrylation procedure is as follows:
Acid solubilized Jellyfish Collagen (Jellagen Ltd).
Hydrochloric acid (HCl, Sigma Aldrich).
Acetic acid (AcA, Sigma Aldrich).
Aminoethyl methacrylate (AM, Sigma Aldrich).
2-(N-morpholino)ethanesulfonic acid (MES, Sigma Aldrich).
1-Ethyl-3-(3-dimethylaminopropyl)carbodiimide (EDC, Sigma Aldrich).
N-Hydroxysuccinimide (NHS, Sigma Aldrich).

Method of Methacrylating Jellyfish Collagen

The method of collagen methacrylation and analysis is described below:
1. 200 mL of acid solubilised jellyfish collagen was placed in dialysis bags and dialysed against 25 L of 10 mM HO (pH 2.0) for 72 hours at 4-8° C.
2. MES buffer was added to the dialysed collagen solution to a final concentration of 50 mM.
3. The collagen solution was adjusted to pH 5.0 through addition of 0.5M NaOH.
4. Aminoethyl methacrylate was added to the collagen solution at the concentration defined for Conditions A, B, or C (see Table 1 below) and the pH was kept constant at pH 5.0.
5. EDC and NHS were added to the collagen solution at the concentrations defined for Conditions A, B, or C (see Table 1 below).
6. The pH of the solution was monitored to ensure that it was kept around pH 5.0 and the solution was incubated with stirring overnight at 4° C. to allow methacrylation to occur.
7. After overnight incubation, methacrylation was stopped by adjusting the solution to pH 2.8-3.6 using HCI.
8. The methacrylated collagen solution was extensively dialysed against 20 mM AcOH for 72 hour at 4-8° C.
9. The methacrylated collagen solution was freeze-dried in preparation for Fourier Transform Infrared (FTIR) analysis (as described below)

TABLE 1
Methacrylation conditions

| | Aminoethyl | | | |
|---|---|---|---|---|
| | methacrylate | EDC | NHS | Temperature |
| Condition | (mg/mL) | (mg/mL) | (mg/mL) | (° C.) |
| A | 25 | 5 | 0.5 | 4 |
| B | 10 | 2.5 | 0.25 | 4 |
| C | 5 | 5 | 0.5 | 4 |

Fourier Transform Infrared (FTIR) Analysis

FTIR spectra of methacrylated vs non-methacrylated collagen biomaterial were produced using an Attenuated total reflectance (ATR) module. Briefly, a small amount of dried collagen material was placed onto the diamond crystal and an average of three scans were taken. The FTIR was carried out with the ATR module using standard methods known in the art.

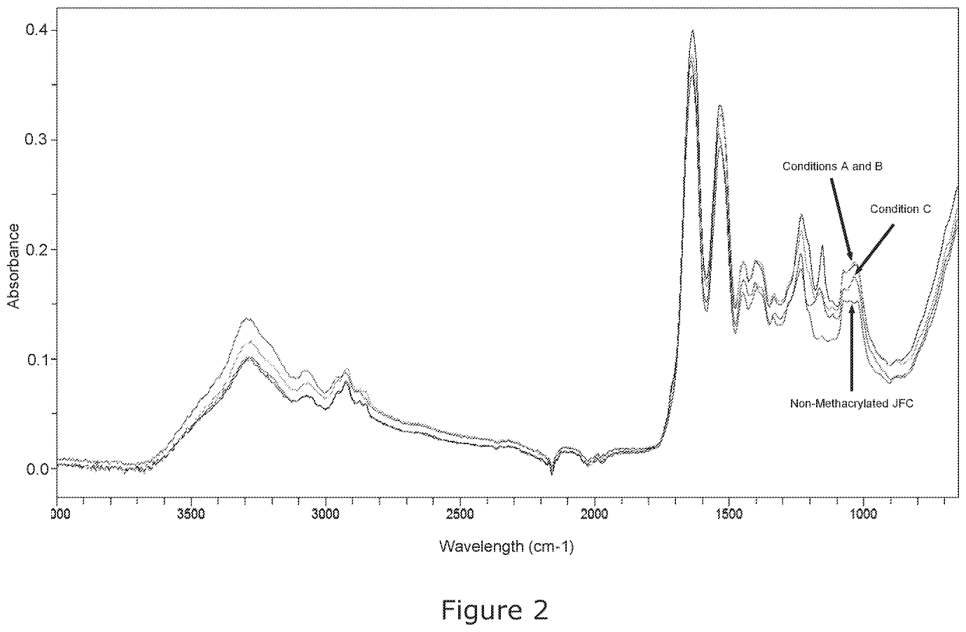

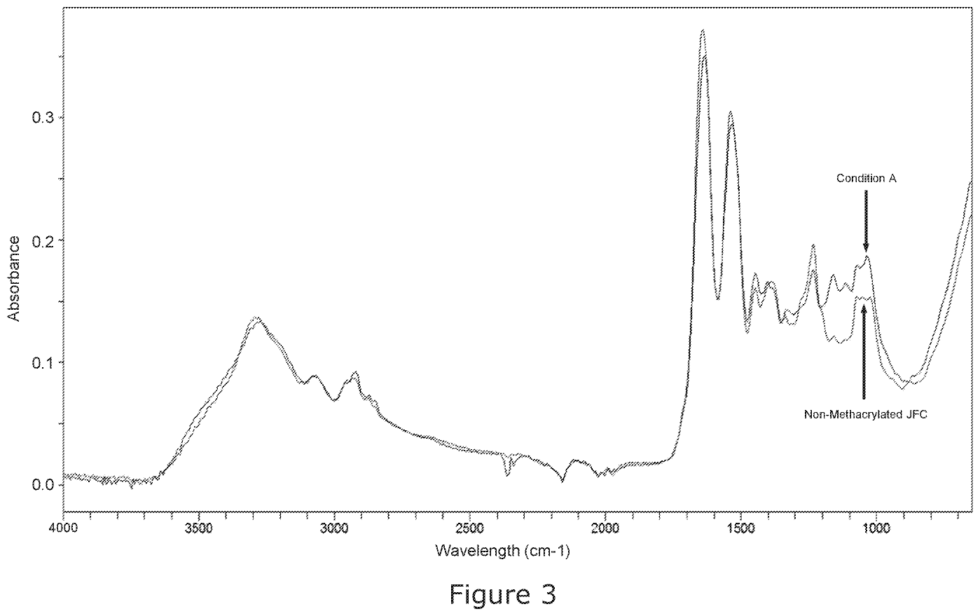

The results of the FTIR analysis of methacrylated and non-methacrylated jellyfish collagen are shown in FIGS. 2 and 3. FIG. 2 shows a comparison of methacrylated jellyfish collagen made according to Condition A, B, or C and non-methacrylated jellyfish collagen. In the FTIR comparison between unmodified jellyfish collagen biomaterial and the methacrylated jellyfish collagens a clear difference can be observed in the FTIR traces for jellyfish collagen made according to each of Conditions A, B, and C at around 1150 cm−1 relative to unmodified control jellyfish collagen, which is indicative of successful modification with methacrylate groups (J. Z. Mbese and P. A. Ajidabe, 2014. Polymers, 6(9): 2332-2344).

FIG. 3 shows a comparison of methacrylated jellyfish collagen made according to Condition A and non-methacrylated jellyfish collagen and further emphasises the characteristic difference observed in the methacrylated jellyfish collagen FTIR traces at a wavelength of around 1150 cm−1 relative to unmodified control jellyfish collagen.

1. A collagen methacrylamide, characterised in that the collagen is derived from a non-mammalian source.

2. The collagen methacrylamide according to claim 1, wherein the nonmammalian source is a jellyfish.

3. The collagen methacrylamide according to claim 1, wherein the jellyfish is selected from the group comprising: Rhizostomas pulmo, Rhopilema esculentum, Rhopilema nomadica, Stomolophus meleagris, Aurelia sp., Nemopilema nomurai, or any combination thereof.

4. The collagen methacrylamide according to claim 1, wherein at least part of the collagen methacrylamide is cross-linked.

5. The collagen methacrylamide according to claim 1, wherein the collagen methacrylamide is in a lyophilised form.

6. The collagen methacrylamide according to claim 1, wherein the collagen methacrylamide is in the form of a hydrogel.

7. The form of collagen methacrylamide according to claim 5, wherein the hydrogel or lyophilised form of collagen methacrylamide further comprises a growth factor, antimicrobial compound and/or a therapeutic pharmaceutical compound.

8. The collagen methacrylamide according to claim 1, wherein the collagen methacrylamide is stable at a temperature of from 15° C. to 80° C.

9. The collagen methacrylamide according to claim 8, wherein the collagen methacrylamide is stable at up to at least 37° C.

10. The collagen methacrylamide according to claim 1 for use in 3D-printing of tissue or cellular scaffolds.

11. The collagen methacrylamide according to claim 1 for use in 3D-printing of tissue models.

12. The collagen methacrylamide according to claim 1 for use in the treatment and healing of wounds.

13. The collagen methacrylamide according to claim 1 for use as a cosmetic.

14. The collagen methacrylamide according to claim 1 for use in regenerative medicine.

15. A method for manufacturing the collagen methacrylamide according to claim 1, wherein the method comprises the following steps:
i) reacting methacrylic acid with a carboxylic acid activating reagent in the presence of a carbodiimide to form a methacrylic acid with an activated carboxylic acid group; and
ii) reacting free amino groups on the collagen with the activated carboxylic acid groups on said methacrylic acid to form a collagen methacrylamide, or
reacting free amino groups on the collagen with aminoethyl methacrylate in the presence of a carboxylic acid activating reagent and a carbodiimide.

16. The method of claim 15, wherein the method further comprises the following steps:
i) removing excess reagents from said collagen methacrylamide;
ii) reacting free carboxylic acid groups on said collagen methacrylamide with a carboxylic acid activating reagent in the presence of a carbodiimide to form a collagen methacrylamide with activated carboxylic acid groups; and
iii) reacting said activated carboxylic acid groups on said collagen methacrylamide with aminoethylmethacrylate in the presence of a carbodiimide to form a collagen methacrylamide amidoethylmethacrylate.

17. The method according to claim 15, wherein the carboxylic acid activating reagent is selected from the group comprising of: N-hydroxysuccinimide (NHS), N-hydroxysulfosuccinimide (Sulfo-NHS), Hydroxybenzotriazole (HOBt), 1-Hydroxy-7-azabenzotriazole (HOAt), pentafluorophenol and methyl N-(triethylammoniumsulfonyl)carbamate.

18. The method according to claim 15, wherein the carbodiimide is selected from the group comprising of: 1-ethyl-3-(3-dimethylamino-propyl) carbodiimide (EDC), N,N′-dicyclohexylcarbodiimide (DHC), N, N′-diisopropylcarbodiimide (DIC), 1-(3-dimethylaminopropyl)-3-ethyl carbodiimide hydrochloride, N-cyclohexyl-N′-(T-morpholinoethyl) carbodiimide-metho-p-toluene sulfonate, N-benzyl-N′-(3′dimethylaminopropyl-carbodiimide hydrochloride, 1-ethyl-3-(3-dimethylaminopropyl) carbodiimide methiodide, N-ethylcarbodiimide hydrochloride, genipin, riboflavin, glutaraldehyde (GD), grape seed extract (GSE) and epigallocatechin-3-gallate.

19. The method according to claim 15, further comprising the step of cross-linking the collagen methacrylamide with a cross-linking agent.

20. The method according to claim 19, wherein the cross-linking agent is poly(ethylene glycol) or light of any wavelength.

21. The method according to claim 15, wherein at least 2% of the collagen free amino acid groups are acrylated.

22. The method according to claim 15, wherein the pH is pH 7.4.

23. A collagen methacrylamide formed by the process according to claim 15.

24. The collagen methacrylamide according to claim 23, wherein the nonmammalian source is a jellyfish.

25. A bio-ink comprising the collagen methacrylamide according to claim 1.
